(19) United States
(12) Patent Application Publication
(10) Pub. No.: US 2012/0322890 A1
(43) Pub. Date: Dec. 20, 2012

(54) Inhibition of the Survival of Bone Cancer by Cyclohexenone Compounds from Antrodia Camphorata

(75) Inventors: Sheng-Yun Liu, New Taipei City (TW); Wu-Che Wen, New Taipei City (TW); Mao-Tien Kuo, New Taipei City (TW)

(73) Assignee: Golden Biotechnology Corporation, New Taipei City (TW)

(21) Appl. No.: 13/597,144

(22) Filed: Aug. 28, 2012

Related U.S. Application Data
(63) Continuation of application No. 12/832,624, filed on Jul. 8, 2010.

(30) Foreign Application Priority Data
Jul. 9, 2009 (TW) 098123273

(51) Int. Cl. A61K 31/122; A61P 35/04

(52) U.S. Cl. 514/690

(57) ABSTRACT

The present invention relates to a novel application of a compound. The compound 4-hydroxy-2,3-dimethoxy-6-methyl-5-(3,7,11-trimethyl-dodeca-2,6,10-trienyl)-cyclohex-2-enone of the invention is isolated and purified from the extracts of Antrodia camphorata, which can be applied for inhibiting the survival of bone cancer cells and be used as a pharmaceutical composition to inhibit the bone tumor growth.

### BACKGROUND OF THE INVENTION

1. Field of the Invention

The present invention relates to a new application for inhibiting cancer cell survival, in particular to the application for inhibiting the survival of bone cancer cells by a compound isolated and purified from Antrodia camphorata.

2. The Prior Arts

Bone cancer such as osteosarcoma, a malignant neoplasm developing predominantly in skeleton system, is the most common type of primary bone cancer which invades to adjacent tissues and organs and is possible to metastasize to other distal organs. It mostly occurs in children and adolescents between 10 and 20 years, and the incidence rate of male is slightly greater than that of female. Although the precise etiological factors of bone cancer remain unclear, it's primary site and prevalence during the rapid growth spurt shows the probable association between bone cancer and the increasing activity of osteoblasts.

Bone cancer arises most frequently at the end of the long bones and wherein approximately 50 percent is in the areas near to knees. Besides, it appears that genetic factors play a role in the etiology of bone cancer and chronic inflammation, radiation or virus infection are also in connection with bone cancer. Symptoms of bone cancer include pain, tumidity, pathology bone fracture, motor function dysfunction, lump and other atypical symptoms such as weight loss, loss of appetite, mild fever or having a sore waist and an aching back, and resulting in a high mortality rate. Hence, bone cancer is a kind of eerie and horrible cancer. In the early ages, amputation is the main surgical treatment strategy for bone cancer and even so the overall 5-year survival rate after the limb-salvage surgery is quite low. As a result of the improvement of chemotherapy and the progress in radiation and surgery, an improvement in overall 5-year survival rate of 70-80% and long-term survival can be achieved until now. However the malignancy of bone cancer still cannot be completely controlled and the chemotherapy drugs used in combination with surgery or radiation all have the effect of myelosuppression.

Thus, it is necessary to develop an effective but less side effect therapeutic substance to apply for the clinic utilization of bone cancer, further to lower the treatment amount of chemotherapy drugs, minimize side effects and achieve higher therapeutic efficacy.

Antrodia camphorata is also known as various names such as Chang-Chih, Ganoderma comphoratum, Antrodia camphorata, Taiwanofungus camphorata, and Camphor Mushroom . . . etc., a genus of Basidiomycoya, Homobasidiomycetes, Aphyllophorales, Polyporaceae, and Antrodia in Fungi, and also a perennial mushroom. It is a Taiwan endemic species of fungi and received its name because it only grows on the inner wall of the hollow material from Taiwan's endemic Lauraceae tree species, Cinnamomum kanehirai. The price of Antrodia camphorata is very high due to the extremely slow growth rate of natural Antrodia camphorata.

The fruiting bodies of Antrodia camphorata are perennial, sessile, hard and woody, which exhale strong smell of sassafras (camphor aroma). The appearances are various with plate-like, bell-like, hoof-like, or tower-like shapes. They are reddish in color and flat when young, attached to the surface of wood. Then the brims of the front end become reversely curled tilting and extending to the surroundings. At the same time, the color turns to be faded red-brown or cream yellow brown, with ostioles all over. This region is of very high medical value.

In traditional Taiwanese medicine, the curative effects of Antrodia camphorata include removing rheumatism, smoothing vitality, nourishing blood, eliminating bruises, benefiting spleen and stomach, lessening accumulation, detoxification, subsiding swelling, sedation and relieving pain, and is used as a great antidote for detoxifying food poisoning, diarrhea, vomiting and pesticide poisoning. Furthermore, it has adjuvant therapeutic effects on liver and stomach dysfunction and the diseases of blood circulation. Antrodia camphorata, like general edible and medicinal mushrooms, is rich in numerous nutrients including polysaccharides (such as β-glucosan), triterpenoids, superoxide dismutase (SOD), adenosine, proteins (immunoglobulins), vitamins (such as vitamin B, nicotinic acid), trace elements (such as calcium, phosphorus and germanium and so on), nucleic acid, agglutinin, amino acids, steroids, lignins and stabilizers for blood pressure (such as antrodia acid) and so on. These physiologically active ingredients are believed to exhibit effects such as: anti-tumor activities, increasing immuno-modulating activities, anti-allergy, anti-bacteria, anti-hypertension, decreasing blood sugar, decreasing cholesterol, etc.

Triterpenoids are the most studied components among the numerous compositions of Antrodia camphorata. Triterpenoids are the summary terms for natural compounds, which contain 30 carbon atoms with the pent- or hex-acyclic structures. The bitter taste of Antrodia camphorata is from the component of triterpenoids. Three novel ergostane-type triterpenoids (antcin A, antcin B, antcin C) were isolated by Cherng et al. from the fruiting bodies of Antrodia camphorata (Cherng, I. H., and Chiang, H. C. 1995. Three new triterpenoids from Antrodia cinnamomea. J. Nat. Prod. 58:365-371). Three new compounds zhankuic acid A, zhankuic acid B and zhankuic acid were extracted from the fruiting bodies of Antrodia camphorata with ethanol by Chen et al. (Chen, C. H., and Yang, S. W. 1995. New steroid acids from Antrodia cinnamomea,—a fungus parasitic on Cinnamomum micranthum. J. Nat. Prod. 58:1655-1661). In addition, Cherng et al. also found three other new triterpenoids from the fruiting bodies of Antrodia camphorata, which are sesquiterpene lactone and 2 biphenyl derived compounds, 4,7-dimethoxy-5-methy-1,3-benzodioxole and 2,2′,5,5′-teramethoxy-3,4,3′,4′-bi-methylenedioxy-6,6′-dimethylbiphenyl (Chiang, H. C., Wu, D. P., Chemg, I. W., and Ueng, C. H. 1995. A sesquiterpene lactone, phenyl and biphenyl compounds from Antrodia cinnamomea. Phytochemistry. 39:613-616). In 1996, four novel ergostane-type triterpenoids (antcins E and F and methyl antcinates G and H) were isolated by Cherng et al. with the same analytic methods (Chemg, I. H., Wu, D. P., and Chiang, H. C. 1996. Triteroenoids from Antrodia cinnamomea. Phytochemistry. 41:263-267). And two ergostane related steroids, zhankuic acids D and E together with three lanosta related triterpenes, 15 alpha-acetyl-dehydrosulphurenic acid, dehydroeburicoic acid, and dehydrosulphurenic acid were isolated by Yang et al. (Yang, S. W., Shen, Y. C., and Chen, C. H. 1996. Steroids and triterpenoids of Antrodia cinnamomea—a fungus parasitic on Cinnamomum micranthum. Phytochemistry. 41:1389-1392).

Although Antrodia camphorata extracts were reported to have the above mentioned effects from the previously published experimental results, and the several compounds were analyzed and identified successfully, further works are needed to identify the effective compound to inhibit cancer growth and thus to contribute beneficial effects on cancer therapy such as the treatment and prevention of bone cancer.

### SUMMARY OF THE INVENTION

In order to identify the anti-cancer compounds from the extracts of Antrodia camphorata, the compound of the formula (1) was isolated and purified in the present invention,

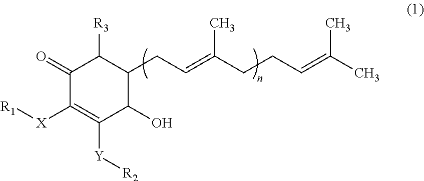

wherein X and Y can be oxygen, nitrogen or sulfur, R₁, R₂ and R₃ are each a hydrogen atom, methyl or (CH₂)ₘ—CH₃ and m=1-12; n=1-12.

A preferred compound of the general formula (1) is 4-hydroxy-2,3-dimethoxy-6-methyl-5-(3,7,11-trimethyl-dodeca-2,6,10-trienyl)-cyclohex-2-enone as shown in formula (2), with molecular formula of C₂₄H₃₈O₄, appearance of pale yellow powder and molecular weight of 390.

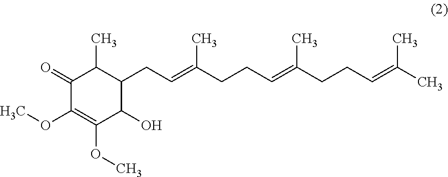

Cyclohexenone compounds having the structures of formula (1) and formula (2) are purified from aqueous extraction or organic solvent extraction of Antrodia camphorata. The organic solvents used include, but not limited to, alcohols such as methanol, ethanol or propanol, esters such as ethyl acetate, alkanes such as hexane, or halogenated alkanes such as chloromethane, chloroethane. Among them, alcohol is preferred, and ethanol is particularly preferred.

Cyclohexenone compounds of the present invention are applied in inhibiting the survival of cancer cells, which can further be used as a pharmaceutical composition for treating cancer and to enhance the cancer therapeutic effects. The compounds of the invention can be applied in inhibiting the survival of bone cancer cells, which result in delaying the growth of the cancer cells and suppressing proliferation of the cancer cells, and further inhibiting cancer deterioration. The preferred compound is 4-hydroxy-2,3-dimethoxy-6-methyl-5-(3,7,11-trimethyl-dodeca-2,6,10-trienyl)-cyclohex-2-enone of the formula (2).

On the other hand, the compounds of formula (1) and/or formula (2) in the present invention can be incorporated into pharmaceutical compositions for treating bone cancer to inhibit the survival of cancer cells. The pharmaceutical compositions include not only the compounds of formula (1) and/or formula (2), but also the pharmaceutically accepted carriers. Examples of such carriers include, but are not limited to, excipients such as water, fillers such as sucrose or starch, binders such as cellulose derivatives, diluents, disintegrants, absorption enhancers or sweeteners. The pharmaceutical composition can be manufactured through mixing the compounds of formula (1) and/or formula (2) with at least one of the carriers by means of conventional methods known in the pharmaceutically technical field, which can be formulated in the form of, but are not limited to, powder, tablets, capsules, pellets, granules or other liquid formulation.

The present invention is further explained in the following embodiment illustration and examples. Those examples below should not, however, be considered to limit the scope of the invention, it is contemplated that modifications will readily occur to those skilled in the art, which modifications will be within the spirit of the invention and the scope of the appended claims.

### DETAILED DESCRIPTION OF THE PREFERRED EMBODIMENT

The aqueous or organic solvent extracts of Antrodia camphorata were subjected to high-performance liquid chromatography (HPLC) for isolation and purification. Each fraction was recovered and applied to anti-cancer assay. The potent fractions with anti-cancer effects were analyzed for the composition and further assayed against bone cancer cells. The above approach then led to the identification of compounds of formula (1) and formula (2) in inhibiting the survival of bone cancer cells.

The compound 4-hydroxy-2,3-dimethoxy-6-methyl-5-(3,7,11-trimethyl-dodeca-2,6,10-trienyl)-cyclohex-2-enone of the formula (2) is explained below as an example for the present invention. The anti-cancer effects of 4-hydroxy-2,3-dimethoxy-6-methyl-5-(3,7,11-trimethyl-dodeca-2,6,10-trienyl)-cyclohex-2-enone was assessed using 3-(4,5-dimethylthiazol-2-yl)-2,5-diphenyltetrazolium bromide (MTF) assay according to the anti-cancer drug screening model of National Cancer Institute (NCI) to analyze survival rates on bone cancer cell line MG-63. These assays have proved that cyclohexenone compounds from Antrodia camphorata decreased the survival rates of bone cancer cell line MG-63, and simultaneously showed low half inhibition concentration (IC₅₀) value. Therefore, cyclohexenone compounds from Antrodia camphorata can be used for inhibiting the survival of bone cancer cells and further be applied for the treatment of bone cancer. The details of the examples are described as follows:

### Example 1

Isolation of 4-hydroxy-2,3-dimethoxy-6-methyl-5-(3,7,11-trimethyl-dodeca-2,6,10-trienyl)-cyclohex-2-enone

One hundred grams of mycelia, fruiting bodies or mixtures of both from Antrodia camphorata were placed into a flask. A proper amount of water and alcohol (70-100% ethanol solution) was added into the flask and were stirred at 20-25° C. for at least 1 hour. The solution was filtered through both a filter paper and a 0.45 μm membrane, and then collected as the extract.

The extract of Antrodia camphorata was subjected to High Performance Liquid chromatography (HPLC) analysis. The separation was performed on a RP18 column using a mobile phase consisted of methanol (A) and 0.1-0.5% acetic acid (B), with the gradient conditions: the ratio of (B) from 95% to 20% 0-10 minutes, from 20% to 10% 10-20 minutes, kept 10% 20-35 minutes, and increased from 10% to 95% 35-40 minutes at the flow rate of 1 ml/min. The column effluent was monitored with a UV-visible detector.

The fractions collected during 25-30 minutes were concentrated to yield 4-hydroxy-2,3-dimethoxy-6-methyl-5-(3,7,11-trimethyl-dodeca-2,6,10-trienyl)-cyclohex-2-enone, a product of pale yellow powder. The analysis of 4-hydroxy-2,3-dimethoxy-6-methyl-5-(3,7,11-trimethyl-dodeca-2,6,10-trienyl)-cyclohex-2-enone showed the molecular formula of C₂₄H₃₈O₄, molecular weight of 390, and melting point of 48° C.˜52° C. Investigation of NMR spectra showed that ¹H-NMR(CDCl₃)δ(ppm)=1.51, 1.67, 1.71, 1.75, 1.94, 2.03, 2.07, 2.22, 2.25, 3.68, 4.05, 5.07, and 5.14; ¹³C-NMR(CDCl₃)δ(ppm)=12.31, 16.1, 16.12, 17.67, 25.67, 26.44, 26.74, 27.00, 39.71, 39.81, 4.027, 43.34, 59.22, 60.59, 120.97, 123.84, 124.30, 131.32, 135.35, 135.92, 138.05, 160.45, and 197.12.

### Example 2

### In Vitro Survival Assay for Anti-Bone Cancer Effects

Inhibiting effects of bone cancer cells by cyclohexenone compounds of Antrodia camphorata from example 1 were assessed according to the anticancer-drug screening model of National Cancer Institute (NCI). The compound 4-hydroxy-2,3-dimethoxy-6-methyl-5-(3,7,11-trimethyl-dodeca-2,6,10-trienyl)-cyclohex-2-enone from example 1 was added into the culture media of bone cancer cell line MG-63 to determine the survival rates. Survival of cell was analyzed using MTT assay. MG-63 cell line was an osteoblast-like fibroblast cell line and derived from human osteosarcoma, which had similar cell type and feature with osteoblast.

MTT assay is commonly used to analyze cell proliferation, survival rate of viable cells and cytotoxicity. MTT (3[4,5-dimethylthiazol-2-yl]-2,5-diphenyltetrazolium bromide) is a yellow dye which can be converted to water-insoluble purple formazan on the reductive cleavage of its tetrazolium ring by the succinate tetrazolium reductase in mitochondria of cells. The amount of formazan produced is used to detect the number of viable cells and calculate the survival rates.

The MG-63 cells were cultivated in Dulbecco's modified Eagle's medium (DMEM) supplemented with 10% fetal bovine serum, 100 IU/ml of Penicillin and 100 mg/ml of Streptomycin at 37° C., 5% CO₂ for 24 hours. Proliferated cells were washed once with PBS, treated with 1× trypsin-EDTA, and centrifuged at 1200 rpm for 5 minutes. The supernatant was removed and the cell pellet was resuspended in 10 ml of fresh medium by gently shaking. Cells were seeded onto 96-well plates. Cells treated with the crude extracts of Antrodia camphorata (total ethanol extracts, not purified) were designed as the control group; and cells treated with 4-hydroxy-2,3-dimethoxy-6-methyl-5-(3,7,11-trimethyl-dodeca-2,6,10-trienyl)-cyclohex-2-enone were designed as the experiment group. Both substrates were added in the concentration of 30, 10, 3, 1, 0.3, 0.1 and 0.03 μg/ml respectively. Cells were cultivated at 37° C., 5% CO₂ for 48 hours. Afterward, 2.5 mg/ml of MTT solution was added to each well and incubated in the dark for 4 hours, followed by the addition of 100 μl of lysis buffer to stop the reaction. The absorbances were measured at 570 nm with an ELISA Reader to determine the survival rates. The half inhibition concentration (IC₅₀) value was also calculated and listed in Table 1.

TABLE 1
Results of in vitro survival assay for inhibition of bone cancer cells

| Sample | IC₅₀ (μg/ml) |
|---|---|
| Experiment group (formula 2) | |
| MG-63 | 2.09 |

Refers to the result of table 1, the IC₅₀ value of 4-hydroxy-2,3-dimethoxy-6-methyl-5-(3,7,11-trimethyl-dodeca-2,6,10-trienyl)-cyclohex-2-enone against MG-63 was 2.09 μg/ml, which was significantly lower than those of total extracts from Antrodia camphorata (data not shown). Therefore 4-hydroxy-2,3-dimethoxy-6-methyl-5-(3,7,11-trimethyl-dodeca-2,6,10-trienyl)-cyclohex-2-enone from Antrodia camphorata can be utilized to inhibit the survival of bone cancer cells.

In summary, the compound 4-hydroxy-2,3-dimethoxy-6-methyl-5-(3,7,11-trimethyl-dodeca-2,6,10-trienyl)-cyclohex-2-enone isolated from Antrodia camphorata according to the present invention can be used to effectively inhibit the survival of human bone cancer cells. The cyclohexenone compounds from Antrodia camphorata won't induce uncomfortable side effects, toxicity or complications when being applied for treating bone cancer. Moreover, these compounds of the invention can also be used concurrently with chemotherapy drugs when treating bone cancer in order to reduce the using amount of chemotherapy drugs as well as decreasing the side effects resulted from chemotherapy drugs. In addition, it can be incorporated into pharmaceutical compositions. The pharmaceutical compositions include not only effective amount of the cyclohexenone compounds from Antrodia camphorata of the present invention, but also the pharmaceutically accepted carriers. Examples of such carriers include, but are not limited to, excipients such as water, fillers such as sucrose or starch, binders such as cellulose derivatives, diluents, disintegrants, absorption enhancers or sweeteners. The composition of the present invention can be manufactured through mixing the compound of cyclohexenone from Antrodia camphorata with at least one of the carriers by means of conventional methods known in the pharmaceutically technical field, and can be formulated in the forms of powder, tablets, capsules, pellets, granules or other liquid formulation, but are not limited to. The purpose for treating bone cancer can then be accomplished.

What is claimed is:

1. A method of inhibiting the survival of bone cancer cells, comprising administering to a subject in need thereof an effective amount of a compound having the following formula: 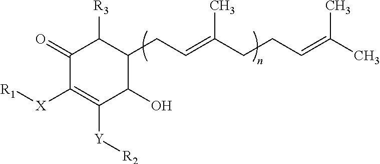
wherein X and Y is oxygen, nitrogen or sulfur, R₁, R₂ and R₃ are each a hydrogen atom, methyl or (CH₂)ₘ-CH₃, and m=1-12; n=1-12.

2. The method as claimed in claim 1, wherein the compound is 4-hydroxy-2,3-dimethoxy-6-methyl-5-(3,7,11-trimethyl-dodeca-2,6, 10-trienyl)-cyclohex-2-enone.

3. The method as claimed in claim 2, wherein the compound is isolated from Antrodia camphorata.

4. The method as claimed in claim 3, wherein the compound is isolated from the aqueous extracts of Antrodia camphorata.

5. The method as claimed in claim 3, wherein the compound is isolated from the organic solvent extracts of Antrodia camphorata.

6. The method as claimed in claim 5, wherein the organic solvents are selected from the group consisting of alcohols, esters, alkanes, and halogenated alkanes.

7. The method as claimed in claim 6, wherein the alcohol is ethanol.

8. The method as claimed in claim 1, wherein the bone cancer cells are from MG-63 cell line.

9. The method as claimed in claim 1, wherein the compound is administered in a form selected from the group consisting of powder, tablet, capsule, pellet, granule and liquor.

10. A pharmaceutical composition for inhibiting the survival of bone cancer cells comprising an active dose of compound as claimed in claim 1 and a pharmaceutically acceptable carrier.

11. The composition as claimed in claim 10, wherein the compound is 4-hydroxy-2,3-dimethoxy-6-methyl-5-(3,7,11-trimethyl-dodeca-2,6,10-trienyl)-cyclohex-2-enone.

12. The composition as claimed in claim 11, wherein the compound is isolated from Antrodia camphorata.

13. The composition as claimed in claim 12, wherein the compound is isolated from the aqueous extracts of Antrodia camphorata.

14. The composition as claimed in claim 12, wherein the compound is isolated from the organic solvent extracts of Antrodia camphorata.

15. The composition as claimed in claim 14, wherein the organic solvents are selected from the group consisting of alcohols, esters, alkanes, and halogenated alkanes.

16. The composition as claimed in claim 15, wherein the alcohol is ethanol.

17. The composition as claimed in claim 10, wherein the bone cancer cells are from MG-63 cell line.
